(19) United States
(12) Patent Application Publication
(10) Pub. No.: US 2006/0184120 A1
(43) Pub. Date: Aug. 17, 2006

(54) Injection apparatus having a plurality of stoppers

(75) Inventor: Russell Anderson, San Diego, CA (US)

Correspondence Address: HELLER EHRMAN LLP, 4350 LA JOLLA VILLAGE DRIVE #700, 7TH FLOOR, SAN DIEGO, CA, 92122, US

(73) Assignee: Artes Medical, Inc., San Diego, CA (US)

(21) Appl. No.: 11/325,618

(22) Filed: Jan. 3, 2006

Related U.S. Application Data
(60) Provisional application No. 60/641,570, filed on Jan. 4, 2005.

(51) Int. Cl. A61M 1/00

(52) U.S. Cl. 604/151

(57) ABSTRACT

An injection apparatus including a first body portion with a first stopper and a second body portion with a second stopper is disclosed. The first body portion is capable of receiving a first material in a portion of the first body portion distal to the first stopper. The second body portion is fluidly coupled to the first body portion, and is capable of receiving the first material in a portion of the second body portion disposed proximally of the second stopper. The second body portion is also capable of receiving a second material in a portion of the second body portion disposed distally of the second stopper.

### CROSS-REFERENCE TO RELATED APPLICATIONS

This application claims the benefit of U.S. Ser. No. 60/641,570, filed Jan. 4, 2005. The entire contents of the aforementioned application is expressly incorporated herein by this reference.

### BACKGROUND OF THE INVENTION

1. Field of the Invention

The present invention relates generally to medical devices and, more particularly, to syringes and needles.

2. Description of Related Art

The term “stress urinary incontinence” refers to a functionally insufficient urinary tract of a patient. In a patient having this condition, the tissue relaxation of the sphincter mechanism, located at the urinary outflow of the bladder into the urethra, can cause a loss of bladder control. Cystoscopes are typically used to study the urethra and bladder and to evaluate a patient's urinary incontinence condition. A typical cystoscope may comprise a tubular instrument equipped with, for example, a visual channel and a working channel, and constructed to be inserted through the urethra for viewing of the urethra and bladder. Treatment of a urinary incontinence condition may comprise the injection of a filler material, such as collagen, into and adjacent to the urinary sphincter muscle at the bladder neck, to thereby bulk up the tissue and assist in the adequate closure of the urinary sphincter.

Acid reflux is a digestive disorder which similarly involves the tissue relaxation of a sphincter mechanism. In the case of acid reflux, which is commonly known as gastroesophageal reflux disease (GERD) or heartburn, the lower esophageal sphincter connecting the esophagus to the stomach begins to malfunction. During proper operation of the lower esophageal sphincter, the lower esophageal sphincter opens to allow food to pass into the stomach and closes to prevent food and acidic stomach fluids from flowing back up into the esophagus. Gastroesophageal reflux occurs when the lower esophageal sphincter is weak or relaxes inappropriately, allowing the stomach's contents to retrograde or flow up into the esophagus. This retrograde flow of gastric contents back into the esophagus, through what should be a one-way valve into the stomach, can damage the esophagus. More particularly, the contents of the stomach are very acidic; and the lining of the stomach is specially designed to cope with the lower pH contents. The esophagus, on the other hand, is not suited for such exposure to highly acidic materials. Thus, when acid retrogrades from the stomach into the esophageal tissues, irritation and inflammation will often result to these tissues.

The severity of tissue damage which can result from gastroesophageal reflux disease can depend on factors such as the dysfunctional level of the lower esophageal sphincter, the type and amount of fluid brought up from the stomach, and the neutralizing effect of the patient's saliva. Another factor, which may affect the severity of a particular gastroesophageal reflux disorder, is the patient's esophageal motility. Lack of esophageal motility can occur through either of two mechanisms. When incomplete emptying of the esophagus into the stomach after ingestion of liquids or solids occurs, the motility of the esophagus can be said to be effected, resulting in esophageal reflux. Also, esophageal reflux can occur when small amounts of gastric contents, which may be refluxed into the lower esophagus, are not rapidly emptied back into the stomach. Delays in the emptying of this material, caused by an esophageal motility disorder, for example, can lead to irritation of the esophageal mucosa and possibly to the sensation of heartburn or the development of esophagitis.

Various tools and instruments have been used in the prior art for the treatment of types of conditions such as the above-mentioned urinary incontinence and acid reflux disease. Gastroscopes are typically used to study the esophagus and to evaluate, for example, a patient's acid reflux condition. A gastroscope typically comprises a flexible, lighted instrument that is inserted through the mouth and esophagus to view the stomach. Similarly, a cystoscope is typically inserted through a patient's urethra to facilitate evaluation of, for example, a urinary incontinence condition.

A material having relatively high viscosity, such as collagen, may be injected into the vicinity of either the lower esophageal sphincter (for acid reflux) or the sphincter of the urethra (for urinary incontinence) to treat either of these disorders. Injection procedures typically involve elongated catheters for delivery of therapeutic materials through body passages to target sites of injection. The force required to deliver a highly viscous material through a delivery lumen of an elongated catheter increases as the average viscosity of the material being delivered increases and as the length of the elongated catheter increases.

### SUMMARY OF THE INVENTION

The invention herein disclosed comprises, according to one embodiment, an injection apparatus having a first body portion with a first stopper disposed therein. The first body portion may be capable of receiving a first material in a portion thereof distal to the first stopper. The embodiment further comprises a second body portion operatively coupled to the first body portion. The second body portion includes a second stopper disposed therein. The second body portion may be capable of receiving a second material in a portion thereon distal to the second stopper and of receiving the first material in a portion thereof proximal to the second stopper.

Another embodiment of the present invention may comprise a syringe adapted for injecting therapeutic material into a patient. The syringe may comprise a first body portion and a second body portion operably coupled to the first body portion. A first stopper may be disposed in the first body portion, the first stopper being attached to a plunger rod capable of moving the first stopper. The embodiment further may include a second stopper disposed in the second body portion.

While the apparatus and method have or will be described for the sake of grammatical fluidity with functional explanations, it is to be expressly understood that the claims, unless expressly formulated under 35 U.S.C. 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. 112 are to be accorded full statutory equivalents under 35 U.S.C. 112.

Any feature or combination of features described herein are included within the scope of the present invention provided that the features included in any such combination are not mutually inconsistent as will be apparent from the context, this specification, and the knowledge of one skilled in the art. For purposes of summarizing the present invention, certain aspects, advantages and novel features of the present invention are described herein. Of course, it is to be understood that not necessarily all such aspects, advantages or features will be embodied in any particular embodiment of the present invention. Additional advantages and aspects of the present invention are apparent in the following detailed description and claims that follow.

### BRIEF DESCRIPTION OF THE FIGURES

FIG. 1 is a simplified cross-sectional drawing of a portion of an injection apparatus employing two stoppers;

FIG. 2 is a cross-section of an injection apparatus having a first stopper and a second stopper with the first stopper abutting the second stopper;

FIG. 3 is an illustration of an injection apparatus having two body portions, one of which comprises an elongate catheter;

FIG. 4 is a cross-sectional diagram of an injection apparatus having two body portions of unequal cross-section with a stopper disposed in each body portion; and

FIG. 5 is a cross-sectional diagram of an injection apparatus having two body portions of unequal cross-section and two stoppers disposed on one of the body portions.

### DETAILED DESCRIPTION OF THE PRESENT INVENTION

Reference will now be made in detail to the presently preferred embodiments of the invention, examples of which are illustrated in the accompanying drawings. Wherever possible, the same or similar reference numbers are used in the drawings and the description to refer to the same or like parts. It should be noted that the drawings are in simplified form and are not to precise scale. In reference to the disclosure herein, for purposes of convenience and clarity only, directional terms, such as, top, bottom, left, right, up, down, over, above, below, beneath, rear, and front, are used with respect to the accompanying drawings. Such directional terms should not be construed to limit the scope of the invention in any manner.

Although the disclosure herein refers to certain illustrated embodiments, it is to be understood that these embodiments are presented by way of example and not by way of limitation. The intent of the following detailed description, although discussing exemplary embodiments, is to be construed to cover all modifications, alternatives, and equivalents of the embodiments as may fall within the spirit and scope of the invention as defined by the appended claims. The present invention may be practiced in conjunction with various injection devices that are conventionally used in the art. For purposes of illustration, the present invention may be adapted to an injection device incorporating a medical injection or injection facilitation apparatus as disclosed in the above-referenced U.S. Pat. No. 6,666,848 (the '848 patent). As another example, an elongated or elongated flexible syringe as described in the above-referenced U.S. Pat. No. 6,929,623 (the '623 patent) may be modified to include aspects of the present invention.

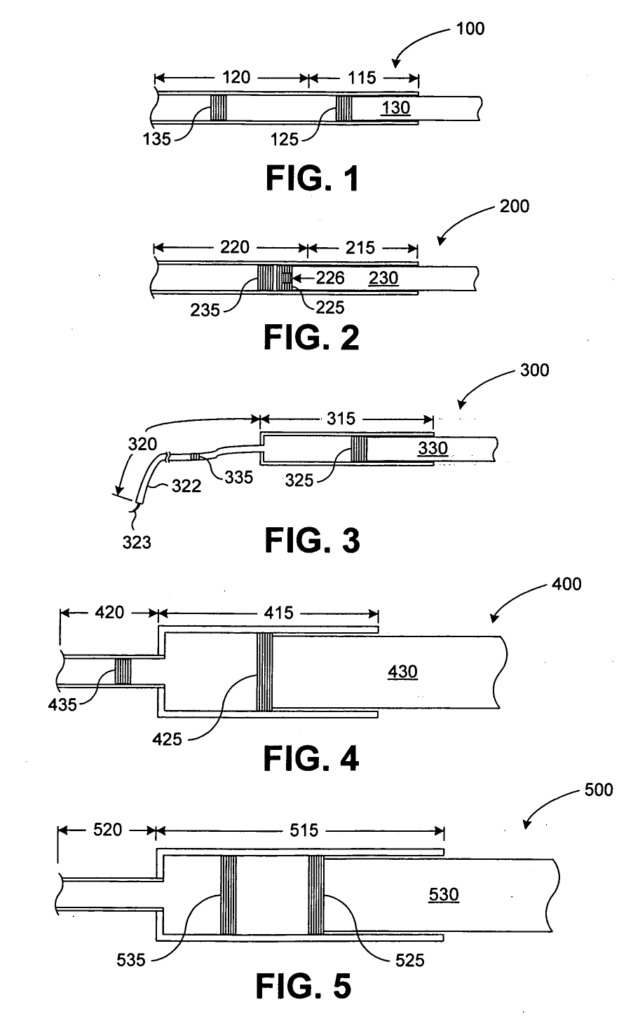

Referring more particularly to the drawings, FIG. 1 is a simplified cross-sectional drawing of a portion of an injection apparatus 100 (e.g., a syringe) comprising a first body portion 115 and a second body portion 120. The first body portion 115 (e.g., chamber) is disposed proximally to the second body portion 120 (e.g., chamber), where it is understood that, as used herein, the term “proximal” means an end or part nearest to an operator of an instrument (e.g., the injection apparatus 100). Conversely, the term “distal” refers to an end or part furthest from the operator. All figures presented herein are oriented with the proximal portions located to the right of distal portions, which, generally, are on the left.

In the exemplary embodiment illustrated in FIG. 1, first and second body portions 115 and 120 form a contiguous tubular structure. First body portion 115 has incorporated therein a first stopper 125 to which may be attached a plunger rod 130 capable of moving the first stopper 125 in response to an applied force in a manner well understood in the art. Second body portion 120 may have incorporated therein a second stopper 135.

In a representative application, a portion of the second body portion 120 that is distal to the second stopper 135 may be adapted to receive a first material to be administered to a patient as a therapeutic agent. Examples of a first material may include a relatively high-viscosity material such as collagen and/or microspheres, as is disclosed in U.S. Pat. No. 5,344,452, the contents of which are expressly incorporated herein by reference. In the same representative application, a portion of the second body portion 120 that is proximal to the second stopper 135 and a portion of the first body portion 115 that is distal to the first stopper 125 may be adapted to receive a second material capable of causing movement of the second stopper 135 in, for example, a distal direction when the first stopper 125 is moved distally. The second material may comprise, for example, a fluid having a relatively low viscosity compared, for example, to a viscosity of the first material. In modified embodiments, the second material may comprise a higher viscosity material, a gel, a flexible solid or semi-solid material, and/or a hard or semi-hardened material such as silicone rubber.

In yet another embodiment, the first stopper 125 may take or resemble a shape (and/or a functionality or the like) of one or more of the distal rod end 79 and the driving piston 80 as shown in FIGS. 1 and 2 of the above-referenced '623 patent, even to the extent, for example, that such element(s) may be disclosed therein as having different function(s). Additionally, the plunger rod 130 in the present invention may correspond to the movable rod 78 in FIGS. 1 and 2 of the '623 patent.

In another implementation, the plunger rod 130 can correspond to a movable plunger 136 of, for example, an injection facilitation apparatus 17 as disclosed in, for example, FIG. 3 of the '848 patent and, in a further implementation, the plunger rod 130 can be removed from the syringe (e.g., injection apparatus 100) and a movable rod (e.g., 113 of FIG. 3 of the '848 patent) of, for example, the injection facilitation apparatus 17 can be configured (e.g., sized and shaped) to operate as the plunger rod of the syringe (e.g., injection apparatus 100).

Indeed, according to another aspect of the present invention, a user can remove plunger rod 130, which may correspond, for example, to a movable rod 113 or a movable plunger 136 of the injection facilitation apparatus 17 as disclosed in FIG. 3 of the '848 patent. In either of those or other instances, when the first stopper 125, which may be attached to the plunger rod 130, is removed, the second stopper 135, according to an aspect the present invention, remains in the injection apparatus 100 to maintain a sealed (e.g., sterile) barrier to the contents disposed distally of the second stopper 235.

The second stopper 135 can be configured similarly to the first stopper 125 in any suitable shape and/or of any suitable material so that the second stopper 135 maintains a sealed (e.g., sterile) barrier to the contents of the second body portion 120 when the plunger rod 130 is removed. For example, the second stopper 135 can take or resemble the shape (and/or functionality, or the like) of element 99 (even to the extent such element may be disclosed therein as having a different function) in FIG. 2 of the '623 patent.

FIG. 2 illustrates another aspect of the present invention wherein an injection apparatus 200 is provided having a first body portion 215, a second body portion 220, a first stopper 225 and a second stopper 235. Elements having a prefix ‘2’ in FIG. 2 may be configured in relation to each other as corresponding elements having a prefix ‘1’ in FIG. 1. First stopper 225 in FIG. 2 has secured thereto a plunger rod 230. According to one embodiment, plunger rod 230 comprises a male threaded portion 226 that screws into a corresponding female threaded portion disposed on a proximal side of the first stopper 225, thereby facilitating convenient removal of the plunger rod 230 from the first stopper 225.

The injection apparatus 200 may be operable to be loaded into the injection facilitation apparatus 17 described in FIG. 3 of the above-referenced '848 patent. The movable rod 113 in the injection facilitation apparatus 17 of FIG. 3 of the '848 patent may be modified to be coupled with or fitted (e.g., threaded) into the first stopper 225 of injection apparatus 200 in place of the plunger rod 230, or in a modified embodiment may be initially formed to comprise a first stopper (e.g., similar to first stopper 225) at its distal end. The first stopper 225 then may, for example, abut with the second stopper 235 as illustrated in FIG. 2. Alternatively, first stopper 225 may be separated from second stopper 235 in a manner similar to the separation of first stopper 125 and second stopper 135 as shown in FIG. 1. Regardless of whether first stopper 225 abuts with second stopper 235 or is separated from second stopper 235, the first stopper 225 may be functionally operable to move the second stopper 235. In accordance with certain scenarios wherein, for example, the plunger rod 230 is removably (e.g., threadably) connected to a proximal end of the first stopper 225, removing the plunger rod 230 may in some implementations attenuate or eliminate a need for a second stopper. In such a case, for example, the second stopper 235 may be reduced in size or absent.

The present invention may be configured in other ways. For example, in the configuration illustrated in the cross-sectional view of FIG. 3, a portion of an injection apparatus 300 is illustrated having a first body portion 315. The first body portion 315 has a relatively large cross-section operable to contain a first stopper 325 that is fitted to a plunger rod 330. The injection apparatus 300 further comprises a second body portion 320 comprising an elongate catheter 322 having a relatively small cross-sectional area and operable to deliver therapeutic material to a patient. The elongate catheter 322 may be fitted with a second stopper 335. The second body portion 320 is operatively coupled to the first body portion 315 by which is meant that the second body portion 320 is fluidly coupled to the first body portion 315, so that material (e.g., a fluid) is able to pass between the first body portion 315 and the second body portion 320. The transition between the first body portion 315 and the second body portion 322 may comprise a rapid transition between diameters as shown or may in modified embodiments comprise one or more gradual transitions between the two diameters to facilitate, for example, relatively low-resistance movement of fluid between the first body portion 315 and the second body portion 322.

A first material may be disposed in a movable portion of the injection apparatus 300 lying proximal to the second stopper 335 and distal to the first stopper 325. A second material may occupy a portion of the elongate catheter 322 lying distal to the second stopper 335. The second stopper 335 thereby may provide a movable barrier (i.e., a seal) between the first material and the second material. The elongate catheter 322 may be fitted with a hollow needle 323 that may, in some applications, be used to inject the second material into a patient.

According to one example, the first material (e.g., a saline solution) has a relatively low viscosity so that relatively little force need be applied to the plunger rod 330 in order to cause distal movement of the first stopper 325. Moving the first stopper 325 distally can increase pressure applied to a proximal side of the second stopper 335, thereby tending to cause the second stopper 330 to move distally, which movement may cause, for example, therapeutic material (e.g., the second material) to pass from the elongate catheter 322 through the hollow needle 323 and into tissue of a patient.

FIG. 4 is a cross-sectional schematic of yet another injection apparatus 400 depicting an embodiment of the present invention. The injection apparatus 400, as illustrated, comprises a first body portion 415 having, for example, a circular cross-section characterized by an inner diameter. The injection apparatus 400 further comprises a second body portion 420 having, for example, a circular cross-section characterized by an inner diameter less than the inner diameter of the first body portion 415. A first stopper 425 is disposed within the first body portion 415, and a second stopper 435 is disposed within the second body portion 420. A plunger rod 430 may be secured to a proximal side of the first stopper 425. In operation, a first material may occupy a portion of the injection apparatus 400 disposed distally of the first stopper 425 and proximally of the second stopper 435. A second material may occupy a portion of the injection apparatus 400 disposed distally of the second stopper 435 in a manner similar to that already described above with reference to FIGS. 1-3.

According to another implementation of the present invention, an embodiment as illustrated in FIG. 5 can comprise an injection apparatus 500 having a first body portion 515 with an inner diameter and a second body portion 520 with an inner diameter that is less than the inner diameter of the first body portion 515. The injection apparatus 500 further comprises a first stopper 525 and a second stopper 535, both of which are disposed in the first body portion 515. A plunger rod 530 may be secured to a proximal side of the first stopper 525. As is the case with the embodiment illustrated in FIG. 4, the embodiment of FIG. 5 may accommodate a first material within a portion of the first body portion 515 located distally of first stopper 525 and proximally of the second stopper 535. A second material (e.g., a therapeutic material suitable for injection into tissue of a patient) may occupy at least part of the second body portion 520 and at least part of the first body portion 515 that is located distally of the second stopper 535.

In view of the foregoing, it will be understood by those skilled in the art that the methods and devices of the present invention can facilitate formation of injection apparatuses. The above-described embodiments have been provided by way of example, and the present invention is not limited to these examples. Multiple variations and modification to the disclosed embodiments will occur, to the extent not mutually exclusive, to those skilled in the art upon consideration of the foregoing description. For example, the first body portions of the examples described herein may have cross-sections smaller than cross-sections of the second body portions. Body portions may have cross-sections that are substantially circular, elliptical, rectangular, or the like, or that take other types of shapes altogether. Transitions between first body portions and second body portions may be abrupt as illustrated herein, or graduated (e.g., tapered) to facilitate, for example, inter-body-portion fluid flow. Bevels and/or chamfers, for example, may be introduced as disclosed, for example, in the above-referenced '848 patent. While exemplary embodiments having two body portions, two stoppers and/or two diameters (e.g., the same or different) have been disclosed herein, other embodiments in accordance with the present invention may comprise, for example, three or more body portions, three or more stoppers and/or three or more diameters (e.g., the same or different). Additionally, other combinations, omissions, substitutions and modifications will be apparent to the skilled artisan in view of the disclosure herein. Accordingly, the present invention is not intended to be limited by the disclosed embodiments, but is to be defined by reference to the appended claims.

What is claimed is:

1. An injection apparatus, comprising:
a first body portion having a first stopper disposed therein, the first body portion being capable of receiving a first material in a portion thereof distal to the first stopper; and
a second body portion operatively coupled to the first body portion, the second body portion having a second stopper disposed therein, the second body portion being capable of receiving a second material in a portion thereof distal to the second stopper and of receiving the first material in a portion thereof proximal to the second stopper.

2. The injection apparatus as set forth in claim 1, wherein:
the first material is a nonviscous fluid; and
the second material is a viscous fluid, relative to the first material.

3. The injection apparatus as set forth in claim 1, wherein a viscosity of the first fluid is less than a viscosity of the second fluid.

4. The injection apparatus as set forth in claim 2, wherein the first material comprises a flexible solid material.

5. The injection apparatus as set forth in claim 4, wherein the flexible solid material comprises a silicone rubber insert.

6. The injection apparatus as set forth in claim 1, wherein:
the first body portion has a substantially circular cross-section characterized by a first inner diameter; and
the second body portion has a substantially circular cross-section characterized by a second inner diameter.

7. The injection apparatus as set forth in claim 6, wherein the first inner diameter and the second inner diameter are substantially equal.

8. The injection apparatus as set forth in claim 6, wherein the first inner diameter is larger than the second inner diameter.

9. The injection apparatus as set forth in claim 6, wherein the first inner diameter is smaller than the second inner diameter.

10. The injection apparatus as set forth in claim 6, wherein the second body portion is a flexible elongate structure.

11. The injection apparatus as set forth in claim 1, further comprising an injection facilitation apparatus comprising a movable rod configured to operate as a plunger rod of the injection apparatus, wherein the plunger rod is attached to a proximal portion of the first stopper.

12. The injection apparatus as set forth in claim 1, further comprising an injection facilitation apparatus comprising a movable rod configured to operate as a plunger rod of the injection apparatus, wherein the plunger rod is removably attached to a proximal portion of the first stopper.

13. The injection apparatus as set forth in claim 12, wherein:
the plunger rod comprises a male threaded portion;
the first stopper comprises a female threaded portion disposed on a proximal side thereof; and
the male threaded portion of the plunger rod is capable of being threaded into the female threaded portion of the first stopper.

14. The injection apparatus as set forth in claim 12, wherein the first stopper does not contact the second stopper and is operable functionally to move the second stopper.

15. The injection apparatus as set forth in claim 12, wherein the first stopper is operable to move the second stopper and the first stopper abuts with the second stopper.

16. A syringe for injecting therapeutic material into a patient, the syringe comprising:
a first body portion;
a second body portion operably coupled to the first body portion;
a first stopper disposed in the first body portion, the first stopper being attached to a plunger rod capable of moving the first stopper; and
a second stopper disposed in the second body portion.

17. The syringe as set forth in claim 16, wherein:
the syringe is capable of receiving a first material into a chamber comprising a part of the first body portion distal to the first stopper and a part of the second body portion proximal to the second stopper; and
the syringe is capable of receiving a second material into a chamber comprising a part of the second body portion distal to the second stopper.

18. The syringe as set forth in claim 17, wherein:
the second material comprises a material to be injected into a patient; and
the first material is a fluid having a viscosity substantially less than a viscosity of the second material.

19. The syringe as set forth in claim 18, wherein application of a distal force to the plunger rod causes distal movement of the second stopper.

20. The syringe as set forth in claim 18, wherein the second material comprises a collagen material.

21. The syringe as set forth in claim 20, wherein the second material comprises microspheres suspended in the collagen material.

22. The syringe as set forth in claim 17, wherein the first material comprises silicone rubber.

23. The syringe as set forth in claim 16, wherein:
the first body portion has a substantially circular cross-section having a first inner diameter;
the second body portion has a substantially circular cross-section having a second inner diameter; and
the second inner diameter is substantially less than the first inner diameter.

24. The syringe as set forth in claim 23, wherein the second body portion comprises an elongate catheter.
